(19) United States
(12) Patent Application Publication
(10) Pub. No.: US 2014/0148678 A1
(43) Pub. Date: May 29, 2014

(54) ELECTRICAL THORACIC SCAN SYSTEM

(71) Applicant: CardioLogic Innovations Ltd., Neve Ilan (IL)

(72) Inventor: Oren DRORI, Binyamina (IL)

(73) Assignee: CardioLogic Innovations Ltd., Neve Ilan (IL)

(21) Appl. No.: 14/090,328

(22) Filed: Nov. 26, 2013

Related U.S. Application Data
(60) Provisional application No. 61/729,931, filed on Nov. 26, 2012.

(51) Int. Cl. A61B 5/053; A61B 5/0408

(52) U.S. Cl. CPC A61B 5/0531; A61B 5/0536; A61B 5/04085
USPC 600/389; 600/393; 600/390; 600/388

(57) ABSTRACT

A method of selecting one or more assay electrodes for use in a device used in an electrical thoracic scan system. The device has a linear multielectrode array, and the method includes providing a device for use in an electrical thoracic scan system. The device includes: a band having an inner surface; and a linear array of electrodes arranged along the length of the band and on the inner surface for contacting the skin surface. Further, each electrode is selectively connectable to a control unit. The method also includes: placing the device on the chest of the subject; designating, as reserve electrodes for potential use, the electrodes making contact with the skin surface; selecting a plurality of assay electrodes from the reserve electrodes; and utilizing the assay electrodes in an electrical thoracic scan process.

### FIELD OF THE INVENTION

The present disclosure relates to the field of instrumentation, as well as related methods, for monitoring and evaluating biophysical measurements in the body. In particular, the disclosure relates to instrumentation for applying probes, such as electrodes, on the thorax of a subject.

### BACKGROUND

Electrical thoracic scans are sensitive to the location of the electrodes that are placed on the patient and inject and/or measure electric currents that pass through or generated by the body. It is also preferable that the electrodes are equally spaced. However, placing these electrodes in a secure way, in the correct locations is a delicate task that requires time, as well as extensive training. Thus, there is a need for a device that enables the correct placement of the electrodes in the correct locations without delicate user instruction (i.e., being user-agnostic). The disclosure below addresses these needs.

One such electrical thoracic scan is electrical impedance tomography (EIT). Pulmonary edema is characterized by a buildup of extracellular fluid in the lungs. It leads to impaired gas exchange and may cause respiratory failure. The condition may have various causes. Pulmonary edema may be cardiogenic, caused by improper heart function, e.g., congestive heart failure (CHF). As such, a reduction in extracellular fluid (e.g., in the lungs) in CHF patients typically indicates an improvement in heart performance. Pulmonary edema may also be caused by an injury to the lungs themselves.

Conventional methods of monitoring pulmonary edema in patients either require expensive equipment and trained personnel (e.g. measuring pulmonary artery and central venous pressure with catheters, measuring blood flow through the mitral annulus and pulmonary veins with doppler echocardiography) or are not very accurate (e.g. monitoring changes in body weight, observing neck vein distension, measuring ankle dimensions). Electrical impedance measurements of the chest have been shown to correlate with the level of retained body water, for example extracellular water in the lungs. Electrical impedance tomography of the chest may be used to monitor the presence and/or severity of pulmonary edema with a high level of accuracy, with less invasiveness to the patient and at lower cost. See, e.g., U.S. Pat. No. 7,096,061.

### SUMMARY OF THE EMBODIMENTS

In a first aspect of the disclosure, the embodiments described herein provide a method of selecting one or more assay electrodes for use in a device for use in an electrical thoracic scan system, the device having a linear multielectrode array, the method comprising the steps of: providing a device for use in an electrical thoracic scan system, the device comprising: a band having an inner surface; a linear array of electrodes arranged equally spaced along the length of the band and on said inner surface for contacting said skin surface; each electrode being selectively connectable to a control unit; placing said device on the chest of the subject; designating, as reserve electrodes for potential use, the electrodes making contact with the skin surface; selecting a plurality of assay electrodes from said reserve electrodes; and utilizing the assay electrodes in an electrical thoracic scan process.

In certain embodiments of the disclosure, the method further comprises the step of disabling the electrodes not in contact with the skin surface.

In certain embodiments of the disclosure, the band partially encircles the chest.

In certain embodiments of the disclosure, the step of placing said device on the chest of the subject comprises the sub-step of wrapping said device fully around the chest of the subject such that: at least a portion of the band fully encircles the chest; the electrodes in the portion of the band fully encircling the chest are in contact with the skin surface; the electrodes in the remaining portion of the band, if present, are not in contact with the skin surface.

In certain embodiments of the disclosure, the step of placing said device on the chest of the subject further comprises the sub-step of: securing the fully wrapped device at the point of band juxtaposition with a connector configured to disable the electrodes in said remaining portion of the band.

In certain embodiments of the disclosure, the device is configured to measure the circumference of the chest of the subject.

Optionally, the circumference of the chest of the subject is measured by a method comprising the steps of: passing an electric current through a section of a wire running through the length of the device corresponding to the portion of the band fully encircling the chest; and calculating the circumference based on the voltage difference through said section of the wire, wherein the wire is characterized by a known resistance per unit length.

Optionally, the outer surface of the device comprises a plurality of optical patterns corresponding to length values, wherein the circumference of the chest of the subject is measured by a method comprising the steps of: reading, via an optical reader, two optical patterns closest to each side of the point of juxtaposition of the device around the chest; and calculating the circumference based on the difference of the length values corresponding to the two optical patterns read by the optical reader.

Optionally, the circumference of the chest of the subject is measured by a method comprising the steps of: providing the number of reserve electrodes; and calculating the chest circumference by multiplying the number of reserve electrodes with the inter-electrode distance.

In certain embodiments of the disclosure, the electrical thoracic scan is selected from the group consisting of: electrical impedance tomography (EIT), parametric EIT (pEIT), electrocardiography (ECG) and body surface mapping.

In certain embodiments of the disclosure, the assay electrodes are selected according to at least one of the specified scheme selected from the group consisting of: a fixed point on the chest along the axial plane of the reserve electroded is set and the assay electrodes are selected at defined intervals from the fixed point around the chest; the assay electrodes are equally spaced; the assay electrodes are symmetrical along the saggital plane of the chest; the assay electrodes are symmetrical along the coronal plane of the chest; the assay electrodes are asymmetrically spaced; the assay electrodes are irregularly spaced; the assary electrode are manually selected.

In certain embodiments of the disclosure, the assay electrodes are designated under control of the microprocessor running a second algorithm comprising the steps of: providing the number of the reserve electrodes R, each of the reserve electrodes being numbered from 1 to R; providing the number of assay electrodes A, such that each assay electrode is designated E₁, E₂, . . . E_{A}; providing a pre-designated set of intervals I₁, I₂, . . . I_{A} between each assay electrode, each interval being a percentage around the perimeter of the chest, such that sum of all intervals equals 100%; designating, as assay electrodes E₁, E₂, . . . E_{A}, each of the reserve electrodes numbered as the closest integer to the product of the interval I and the number of reserve electrodes R or the product of the sum of the intervals I and the number of reserves electrodes R, such that E₁=the closest integer to I₁R; E₂=the closest integer to R(I₁+I₂); E₃=the closest integer to R(I₁+I₂+I₃); . . . E_{A}=the closest integer to R(I₁+I₂ . . . I_{A}).

In certain embodiments of the disclosure, the assay electrodes are equally spaced.

Optionally, the equally spaced assay electrodes are designated under control of the microprocessor running a second algorithm comprising the steps of: providing the number of the reserve electrodes R, each of the reserve electrodes being numbered from 1 to R; providing the number of assay electrodes A; dividing the number of the reserve electrodes R with the desired number of the assay electrodes A to generate interval I; designating each of the reserve electrodes numbered as the closest integer of each multiple of I up to R, as one of the assay electrodes.

Optionally, the equally spaced assay electrodes are designated under control of the microprocessor running a third algorithm comprising the steps of: providing the number of the reserve electrodes R, each of the reserve electrodes being numbered from 1 to R; providing the number of assay electrodes A; providing a gap value G such that the value R−G is divisible by the number of assay electrodes A; dividing (R−G) with the number of assay electrodes A to generate interval I; and designating each of the reserve electrodes numbered as multiples of I+G, up to R, as one of the assay electrodes.

In certain embodiments of the disclosure, the number of electrodes E is more than 50, more than 100, more than 150, more than 200, more than 300, between 50 and 300, between 100 and 300, or between 100 and 500.

In certain embodiments of the disclosure, the device is integrated into an article of clothing.

In certain embodiments of the disclosure, the article of clothing is selected from the group consisting of: a belt, a shirt, a vest and a bra.

In certain embodiments of the disclosure, the electrodes are integrated into a printed circuit board.

In certain embodiments of the disclosure, the device is disposable.

In a second aspect of the disclosure, the embodiments described herein provide a device for use in an electrical thoracic scan system, the device comprising: a band having an outer surface and an inner surface; a linear array of electrodes spaced along the length of the band and on said inner surface for contacting said skin surface; wherein each electrode is selectively connectable to a control unit.

In certain embodiments of the disclosure, the electrical thoracic scan is selected from the group consisting of: electrical impedance tomography (EIT), parametric EIT (pEIT), electrocardiography (ECG) and body surface mapping.

In certain embodiments of the disclosure, the electrical thoracic scan is EIT or pEIT.

In certain embodiments of the disclosure, each electrode is selectively connectable to a current source unit or a voltage measurement unit and said current source unit and voltage measurement unit are independently controlled by a microprocessor such that pairs of the assay electrodes are connectable to the current source, in a controlled sequence, under control of the microprocessor, and the resulting voltages measurements from the remaining assay electrodes are analyzable to generate an impedance image of the subject's chest.

In certain embodiments of the disclosure, at least a portion of the band is configured to fully encircle the chest.

In certain embodiments of the disclosure, the electrodes in the portion of the band fully encircling the chest are in contact with the skin surface; and the electrodes in the remaining portion of the band, if present, are not in contact with the skin surface.

In certain embodiments of the disclosure, the device is integrated into an article of clothing.

In certain embodiments of the disclosure, the article of clothing is selected from the group consisting of: a belt, a shirt, a vest and a bra.

In certain embodiments of the disclosure, the electrodes are integrated into a printed circuit board.

In certain embodiments of the disclosure, the device is disposable.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. The drawings are generally not to scale. Features found in one embodiments can also be used in other embodiments, even if not all features are shown in all drawings. In the accompanying drawings:

FIGS. 1A-B are block diagrams of electrical thoracic scan systems.

FIGS. 2A-B are schematic views of the top and side views the device (strip-type).

FIG. 2C is a schematic side view of the device (loop-type).

FIGS. 3A-D are schematic views of the strip-type device placed on the chest of a subject.

FIGS. 3E-F are schematic views of the loop-type device placed on the chest of a subject.

FIGS. 4A-D are schematic views of various schemes of device placement and reserve electrode selection.

FIGS. 5A-5B are schematic view of the device with a connector.

FIG. 6A is a schematic view of the assay electrodes selected from the total electrodes on a device.

FIG. 6B is a schematic view of the device with a band capable of maintaining the relative distances between the electrodes while the total length is adjustable.

FIGS. 7A-B are schematic views of the assay electrodes including virtual electrodes.

FIG. 8 is a flowchart of a method for selecting electrodes in a multielectrode device for an electric thoracic scan system.

FIG. 9 is a flowchart showing sub-steps of the step of selecting assay electrodes from the reserve electrodes.

FIG. 10 is a flowchart showing sub-steps of the step of selecting equally spaced assay electrodes from the reserve electrodes.

FIG. 11 is a flowchart showing alternative sub-steps of the step of selecting equally spaced assay electrodes from the reserve electrodes.

FIGS. 12A-12D are schematic views of a device with 12 electrodes.

### DETAILED DESCRIPTION

Aspects of the embodiments of the disclosure concern an electrical thoracic scan system, and related devices and methods.

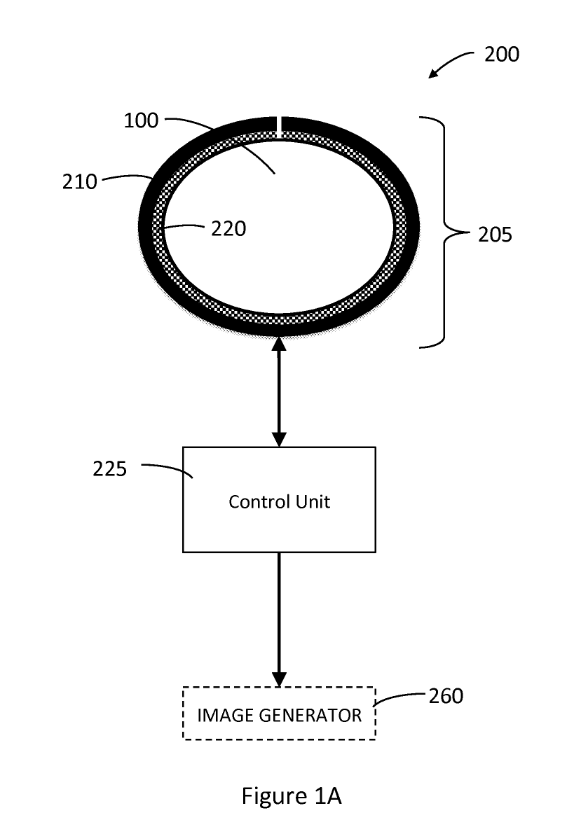

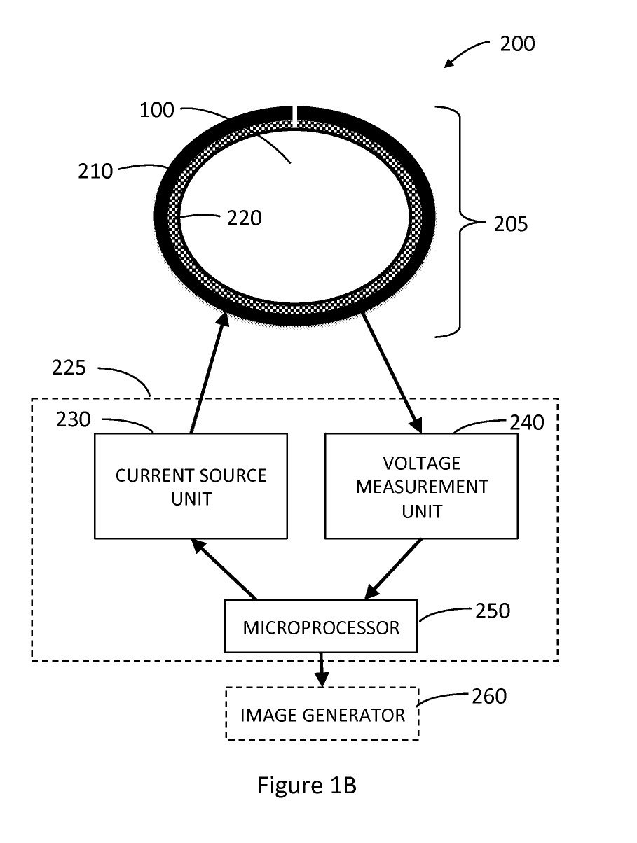

Reference is now made to FIG. 1A, which is a block diagram of an electrical thoracic scan system 200 with a device 205 for receiving electrical signals to a skin surface of the subject's chest 100. The device 205 may further be capable of delivering electrical signals to the skin surface. The device 205 includes a band 210 having an outer surface and an inner surface and a linear array of electrodes 220 arranged equally spaced along the length of the band on said inner surface for contacting said skin surface.

The electrodes 220 may be selectively connectable to a control unit 225. The control unit 225 may control various operations regarding the electrodes 220, including one or more of the following: the selection of a subset of electrodes for actual use in the electrical thoracic scan process; procedures for evaluating the parameters of the electrical contact between the electrodes 220 and the chest skin surface; measurement procedures of the selected electrodes 220 in the electrical thoracic scan process; and stimulation procedures of the selected electrodes 220 in the electrical thoracic scan process. Optionally, the data analysis and the image generation may be executed in a separate image generator 260, e.g., a computer.

With reference to FIG. 1B, the control unit 225 may include a current source unit 230 and a voltage measurement unit 240. The current source unit and voltage measurement unit may be independently controlled by at least one microprocessor, such that each electrode is capable of injecting current to the skin surface of the chest 100, and to measure voltage changes. The microprocessor 250 (or one or more other microprocessors) may be configured to record and analyze the voltage changes measured in at least a portion of the electrodes 220. Optionally, the data analysis and the image generation may be executed in a separate image generator 260, e.g., a computer.

The electrical thoracic scan may be electrical impedance tomography (EIT), electrocardiography (ECG), body surface mapping, and the like. The EIT may be parametric EIT (pEIT).

The EIT or pEIT may be for the purpose of monitoring the level of fluid, e.g., extracellular fluid, in one or more organs of the chest cavity in a subject. The organ may be a lung. The chest impedance image may be for the purpose of monitoring pulmonary edema, which is characterized by a buildup of extracellular fluid in the lungs. The pulmonary edema may be cardiogenic, caused by improper heart function, e.g., congestive heart failure (CHF). Alternatively, the pulmonary edema may be non-cardiogenic and caused by, e.g., an injury to one or both of the lungs.

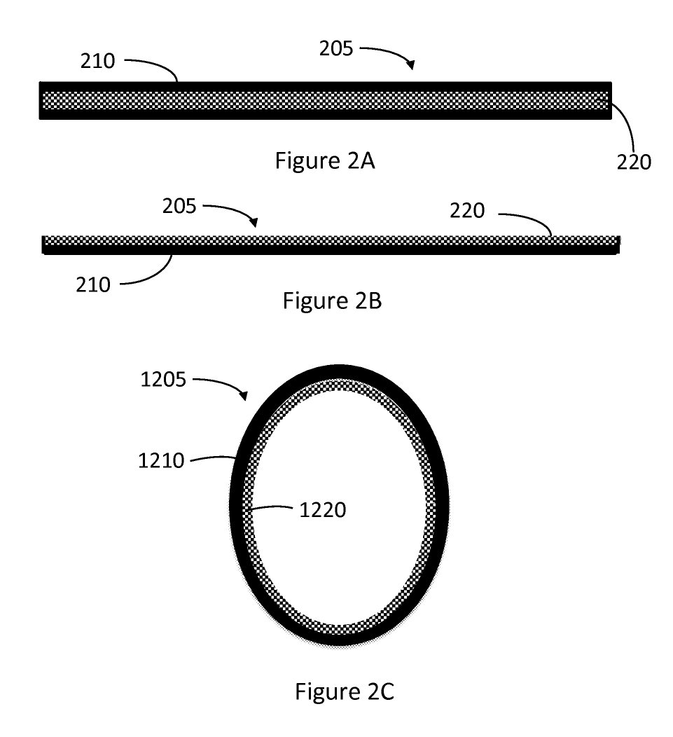

FIG. 2A shows a top view of the device 205 and FIG. 2B shows a side view of the device 205, shaped as a strip. The electrode array 220 may comprise a plurality of individual electrodes. The number of electrodes E may be more than 50, more than 100, more than 150, more than 200, more than 300, between 50 and 300, between 100 and 300, or between 100 and 500, between 200 and 1000, between 300 and 1000, between 400 and 1000, between 500 and 1000, between 400 and 800 or between 300 and 800. Alternatively, the number of electrodes may be between 50 and 6, between 50 and 20, between 40 and 20, between 40 and 10, between 15 and 6, between 20 and 4, between 15 and 4, about 50, about 40, about 30, about 25, about 20, 19, 18, 17, 15, 16, 14, 13, 12, 10, 9, 8, 7, 6, 5 or 4.

The band 210 may be any elongated material that may serve as a substrate for attaching an array of electrodes 220 and other components as needed, and for enabling contact of at least a portion of the electrodes 220 to the skin surface. As such, the band may be a string, a chain, a strip, or the like. The material of the band 210 may be any material that is appropriate for placing on or around the chest of a subject., e.g., fabric, plastic, rubber, metal or a combination thereof. At least a portion of the inner surface of the band may further comprise an adhesive material to aid in the stability of placement of the device 205. The band 210 may be linear in shape or shaped as a loop.

The device 205 may be integrated into an article of clothing, such as a belt, a shirt, a vest, a bra, or other articles of clothing that may be worn around the chest.

The electrodes 220 may be integrated into a printed circuit board (PCB).

The device 205 may be disposable.

Reference is now made to FIG. 2C, which is a schematic diagram showing the side view of an alternative loop-shaped device 1205, including a loop-shaped band 1210 and a linear array of electrodes 1220. The various options described for system 200 and device 205, as described with reference to FIGS. 1A-2B, are also options for the loop-shaped device 1205.

### Placement of the Device on the Chest and the Designation of the Reserve Electrodes

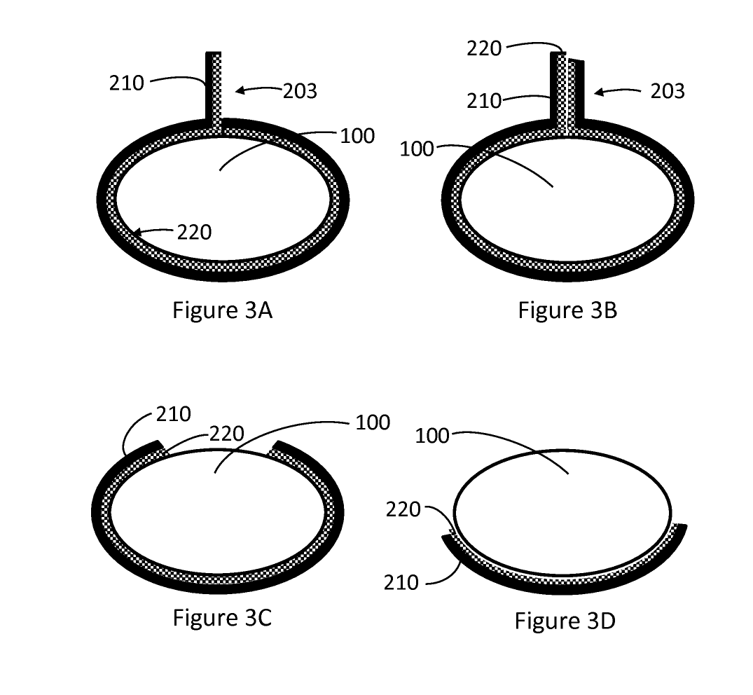

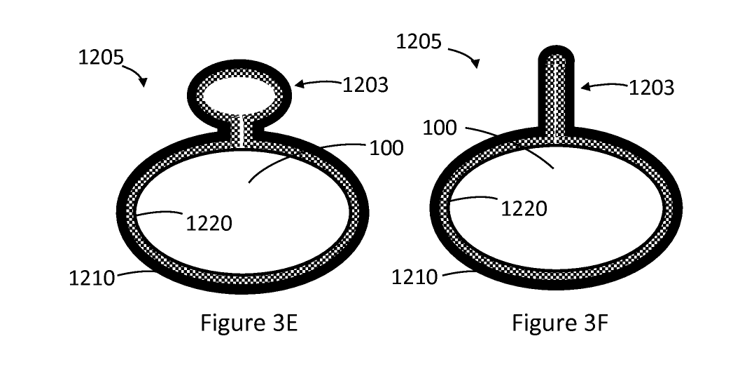

Reference is now made to FIGS. 3A-D, which is a schematic diagram of various configurations in which the device 205 may be placed on the chest 100 of the subject. The device 205 may fully encircle the chest, as shown in FIGS. 3A and 3B, or partially encircle the chest, as shown in FIGS. 3C and 3D. “Placing” the device 205 may be achieved by hanging (if incorporated into an article of clothing), sticking (if at least a portion of the inner surface of the band 210 has an adhesive surface), wrapping (if shaped as a long strip), resting on the chest or back of the subject and held in place via gravity, or other methods that would occur to a skilled practitioner.

The device 205 may be placed on the chest such that the device 205, and thus the electrode array 220, fully encircles the chest once, but not more. In this way, a single ring of electrodes 220 is formed around the chest, contacting the skin thereof. If the device 205 is longer than the circumference of the subject's chest, then there will be at least one remaining portion 203 of the device 205. The electrodes corresponding to the portion of the device contacting the skin may be designated as reserve electrodes that may subsequently selected as assay electrodes for use in the electrical thoracic scan process. This remaining portion 203 of the device 205 may be positioned such that the corresponding electrodes 220 do not make contact with the skin surface of the chest. The electrodes 220 corresponding to the remaining portion 203 may be designated as non-selected electrodes. Typically, if the device is shorter than the circumference of the subject's chest, then the device 205 is positioned such that its entire length makes contact with the skin surface of the subject's chest, with all of the electrodes 220 being designated as reserve electrodes.

FIGS. 3E-F shows the loop-shaped device 1205 placed around the chest of a subject 100. Typically, the circumference of the band 1210 is selected such that it is capable of fully encircling the chest 100 of most subjects regardless of their girth. Alternatively, the device 1205 may be provided in various sizes. If the circumference of the device 1205 is longer than the circumference of the subject's chest 100, then there will be a remaining portion 1203 of the device 1205. This remaining portion 1203 of the device 1205 may be positioned such that the corresponding electrodes 1220 do not make contact with the skin surface of the chest. Further, the electrodes 1220 corresponding to the remaining portion 203 may be designated as non-selected electrodes. The electrodes 1220 corresponding to the portion of the device 205 contacting the skin may be designated as reserve electrodes that may subsequently be selected as assay electrodes for use in the electrical thoracic scan process.

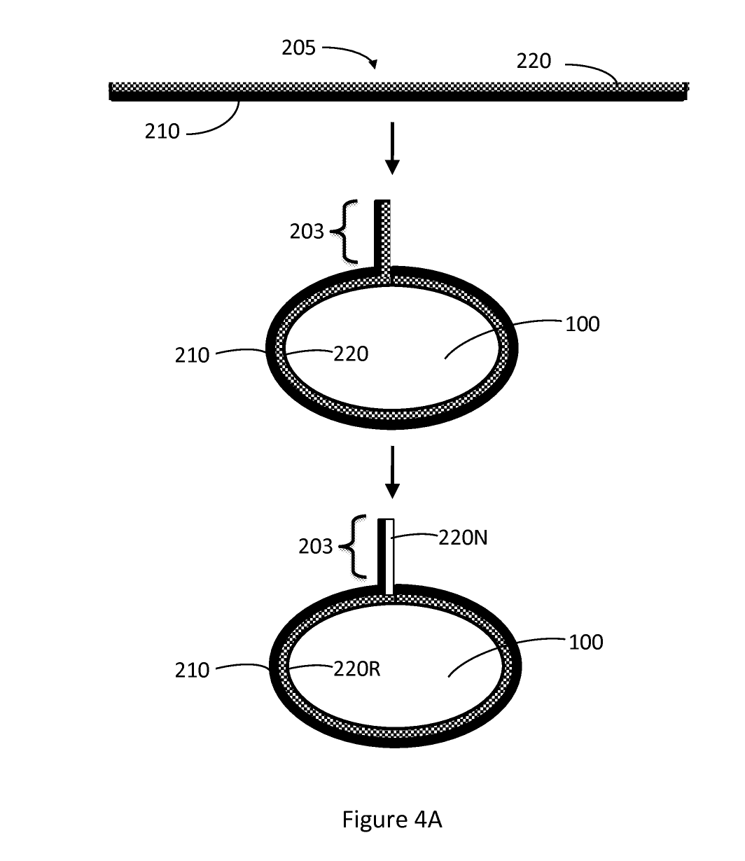

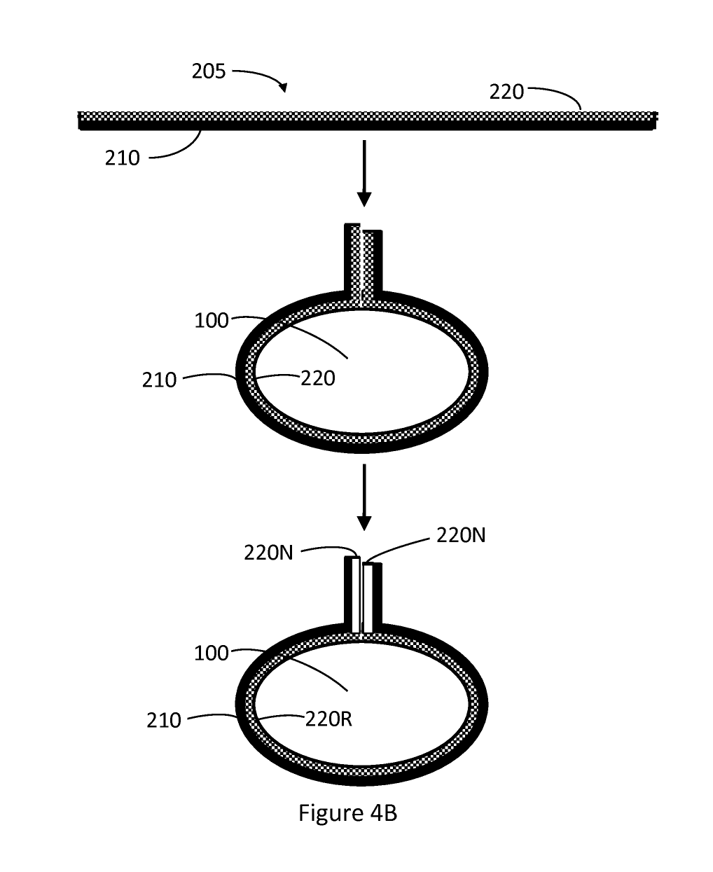

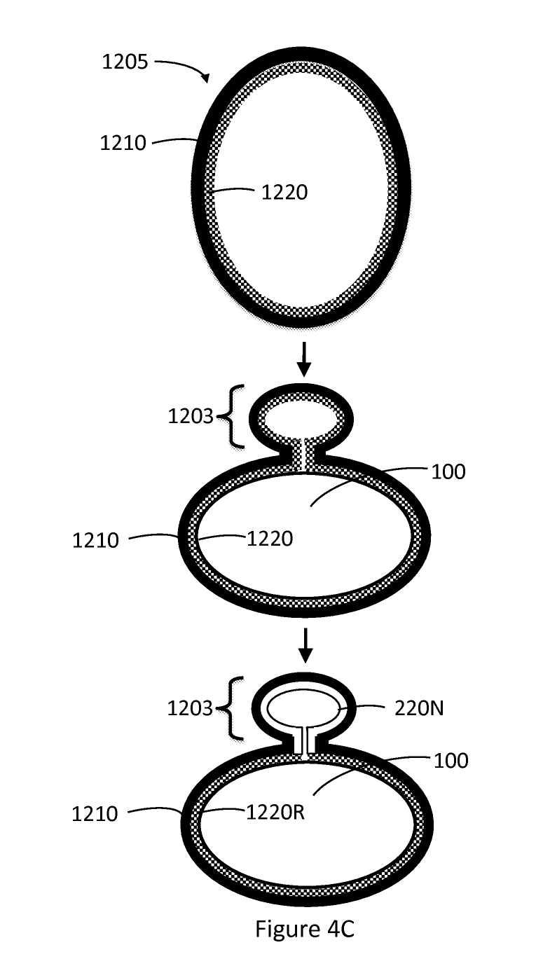

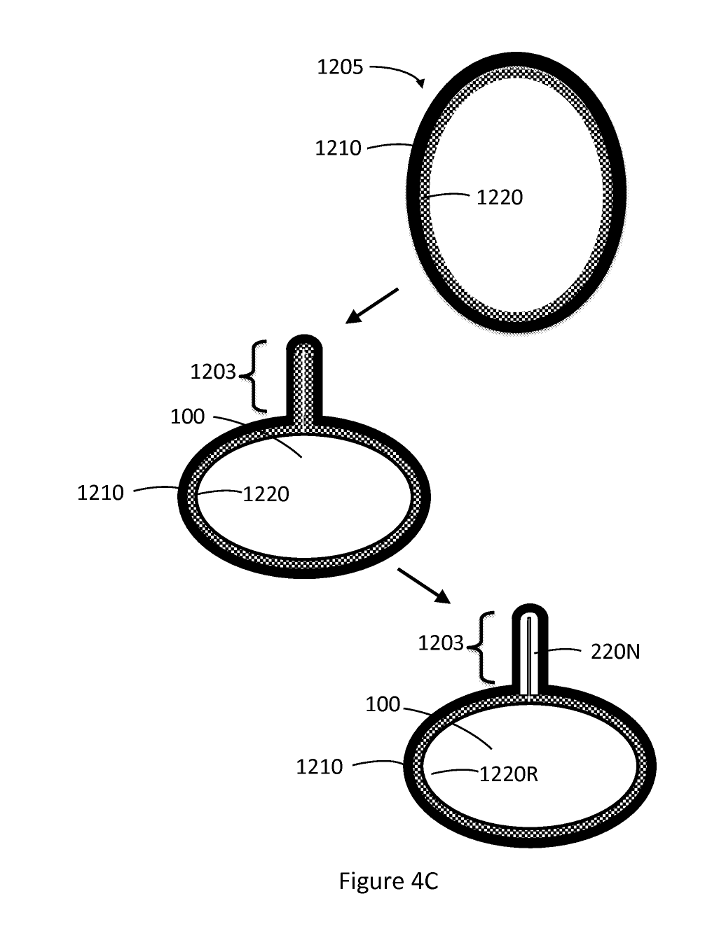

FIGS. 4A-D are schematic drawings showing various examples of the progression from the placement of the device on the chest to the designating of the reserve electrodes. FIGS. 4A-B shows, in the strip-shaped device 205, the process of the electrodes placed around the chest being designated at reserve electrodes 220R, with the electrodes corresponding to the remaining portion 203 being designated as non-selected electrodes 220N. FIGS. 4C-D shows an equivalent process in the loop shaped device 1205.

The process of reserve electrode designation may be accomplished through various mechanisms that may be automatic, manual or a combination thereof.

For an example of an automatic reserve electrode designation, each electrode 220 may be tested for the presence of one or more skin contact signatures, i.e., characteristic electrical properties of an electrode 220 contacting a human skin surface such as resistance, capacitance and/or the like. Such properties may be tested by, e.g., injecting current in an electrode 220 and measure the resulting voltage from the same electrode 220 or a neighboring electrode 220. The electrodes 220 that produce voltage measurements that match the skin contact signatures may then be designated reserve electrodes. This automatic designation process may be a segment-based test, designating defined segments of the electrode array 220 as skin contacting or non-skin-contacting. For example, the reserve electrode designation algorithm may define the electrodes corresponding to one segment of the electrode array 220 having at least 90%, at least 95% or at least 98% of the electrodes pass the skin contact test as the reserve electrodes 220R. The algorithm may then designate the remaining segment(s) as the remaining portion(s) 203 and designate the corresponding electrodes 220 as non-selected electrodes. Alternatively or in addition, the algorithm may define at least one remaining portion 203 as a segment of the electrode array 220 having almost no electrodes pass the skin contact test as the non-selected electrodes 220N. “Almost no electrodes pass the skin contact test” may mean that within the segment of the electrode array 220, the percentage of electrodes passing the skin contact test is be less than 20%, less than 10%, less than 5%, less than 2% or less than 1%.

There are various methods for manual designation of reserve electrodes. As one example, the device 205 may comprise a series of switches along its length that are configured to demarcate the position between the skin contacting and non-skin contacting portions of the device 205. The switches may be configured to be toggled by hand, or with a switch activator. The switch activator may be a tool that enables toggling of the switch, in cases where the switch is designed to be too small to be accurately toggled by hand, or designed such that they cannot be toggled by hand (e.g., in order to prevent accidental switch toggling).

Alternatively, a switch activator may be integrated into a connector that attaches to the two portions of the device 205 that juxtapose after encircling the chest 100.

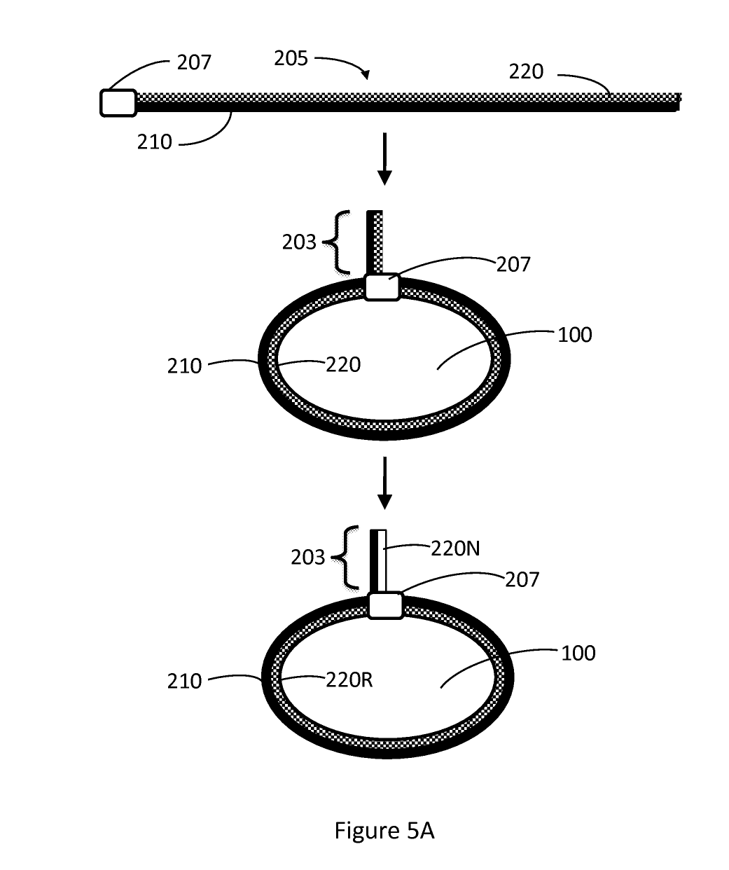

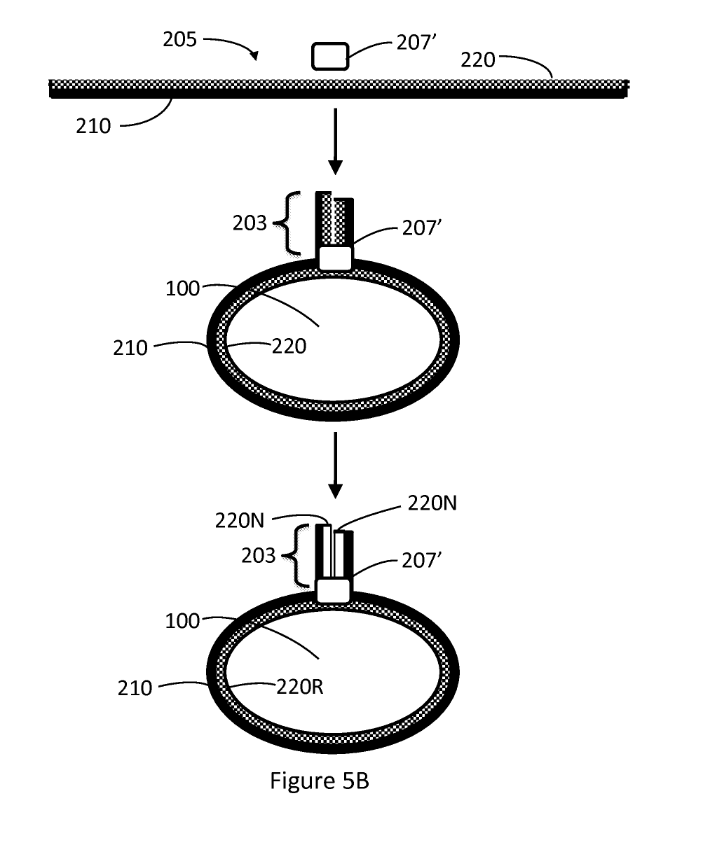

Reference is now made to FIGS. 5A-5B, which is a schematic diagram of the device 205 further including a connector 207. With reference to FIG. 5A, the connector may be integrated into the device 205, for example, attached to a first end of a strip-shaped band 210, and configured so that it connects the first end of the band 210 to another portion of the band 210. The connector 207 may be used to secure the placement of the device 205 on the subject's chest 100. Typically, the device 205 is wrapped around the subject's chest 100, and the connector 207, attached to the first end of the band 210, connects to the portion of the device 205 that first juxtaposes with said first end of the band 210 after encircling the subject's chest 100. Concurrently, the connector 207 may serve to designate as the reserve electrodes 220R the electrodes corresponding to the portion of the device 205 between the first end of the band 210 and the location on the band where the connector 207 is secured.

With reference to FIG. 5B, the connector 207′ may be a separate component that is configured attach to two portions of the device 205, e.g., at the point where they first juxtapose after encircling the subject's chest 100. The connector 207′ may further function as a part of a mechanism for designating the electrodes 220 in the remaining portion 203 of the device as non-selected electrodes 220N and designating as the reserve electrodes 220R the electrodes corresponding to the portion of the device 205 between the point of juxtaposition.

The above-described use of the connector 207 (or 207′) is similarly applicable for use with the loop-shaped device 1205.

Alternatively, the switch activation may be controlled through a separate switch control interface (not shown), and the user may select the desired electrodes as the reserve electrodes, e.g., all electrodes from electrode x to electrode y. The switch control interface may be incorporated into the control unit 225 or image generator 260 (as shown above in FIG. 1A). Alternatively, the switch control interface may be incorporated into a separate device (not shown) connected through a wire or wireless connection to the device 205.

In addition to the reserve electrode designation process describe above, the reserve electrode designation process may further include a process of selecting reserve electrodes at regular intervals. Regular intervals may be intervals such as: every other electrode, even electrodes, odd electrodes, every 5 electrodes, every 10 electrodes, and the like. Such a selection process may be executed through, e.g., the switch control interface.

The reserve electrode designation may operate in one of two modes. The default state of the electrodes 220 may be an active state, and the process of reserve electrode designation may include disabling the non-selected electrodes 220N, while maintaining the reserve electrodes 220R in the active state. Alternatively, the default state of the electrodes 220 may be an inactive state, and the process of reserve electrode selection may include putting the reserve electrodes into a ready state and maintaining the non-selected electrodes 220N in the inactive state.

Preferably, each reserve electrode making contact with the skin surface makes electrical contacts having approximately the same electrical properties (e.g., resistance, capacitance, etc.) The contour of the chest may present challenges in ensuring that, in the portions of the device 205 making contact with the skin surface, the corresponding electrodes are in electrical contact with the skin surface. Regardless of the method of reserve electrode designation, device 205 may comprise various mechanisms to ensure or encourage proper electrical contact to be made between the skin surface and the electrodes 220. The mechanisms may be any one or a combination of the following:
The band 210 may comprise a foam that allows the inner surface of the device 205 to form itself around the contours of the skin surface.
Each electrode 220 may be supported by a spring or a piston.
The device 205 may further comprise a conductive gel layer on its inner surface, such that the gel is situated between each of the electrodes 220 designated as reserve electrodes and the skin surface.
The device 205 may further comprise an adhesive layer on its inner surface.

The process of reserve electrode designation may also include the determination of the chest circumference, which may be useful in the analysis of subsequent voltage measurements. The chest circumference may be separately acquired through various methods known in the art, e.g., wrapping a tape measure near or at the location of the device 205. Alternatively (provided that the device 205 fully encircles the chest), the band 210 may incorporate a tape measure pattern on its outer surface, such that a user (the subject or a separate user applying the device to the subject) may utilize the tape measure pattern to acquire the chest circumference.
The circumference may be measured by automatic or semi-automatic means. The band may include, through it's length, a wire of known resistance per unit length. Once the device is placed around the chest, an electric current may be passed through the section of the wire that corresponds to the part of the device that is contacting the chest. By measuring the voltage, the length of the wire through which the current passed, which is equivalent to the chest circumference, may be calculated. Alternatively or in addition, there may be an optical pattern such as a bar code on the outer surface of the device corresponding to length values, noting the distance along the length of the device. An optical reader device may be configured to read the two optical patterns closest to each side of the point of juxtaposition of the device around the chest and calculate the circumference. The circumference may be calculated based on the difference of the length values corresponding to the two optical patterns read by the optical reader. Alternatively or in addition, the control unit may calculate the chest circumference based on the number of reserve electrodes, based on the reserve electrodes being spaced along the length of the device with a constant inter-electrode distance. The circumference may be calculate by multiplying the multiplying the number of reserve electrodes with the inter-electrode distance.

Once acquired, the chest circumference may be entered into the electrical thoracic scan system through a user interface, to be provided to one or more microprocessor or image generation device. As a further alternative (provided that the device 205 fully encircles the chest), the chest circumference may be calculated automatically once the reserve electrodes are determined. Because the electrodes 220 are equally spaced, the chest circumference may be calculated as the number of electrodes 220 determined to be a reserve electrode R multiplied by the inter-electrode distance (e.g., the distance between the center of two adjacent electrodes).

### Assay Electrode Designation

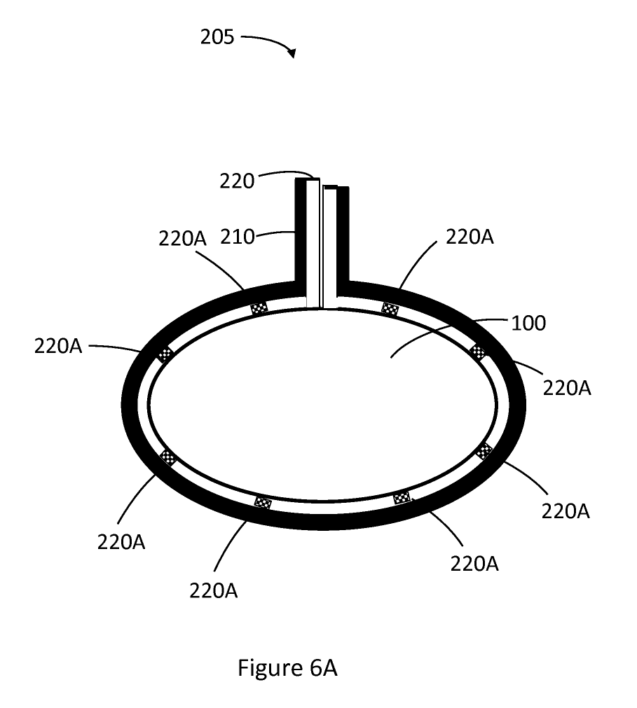

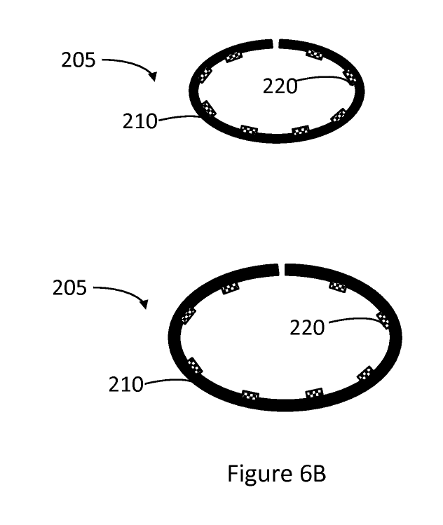

Referring to FIG. 6A, the device 205 may designate a portion of the electrodes 220 as assay electrodes 220A, for use in the electrical thoracic scan process. FIG. 6 is a schematic diagram of the device 205 with a subset of electrodes from the electrodes 220 designated as assay electrodes 220A. The designation of the assay electrodes 220A may be a two-step process. First, as described above, the device 205 may be configured to designate the electrodes 220 corresponding to the portion of the device 205 in contact with the skin surface as reserve electrodes,. Second, the device 205 may further be configured to select a subset out of the reserve electrodes as assay electrodes 220A.

The number of assay electrodes 220A may be, e.g., between 300 and 100, between 200 and 100, between 200 and 50, between 100 and 50, between 100 and 25, between 50 and 25, between 50 and 10, between 32 an 16, between 50 and 4, between 25 and 4, between 15 and 4, about 300, about 250, about 200, about 150, about 100, about 90, about 80, about 70, about 60, about 50, about 45, about 40, about 35, about 32, about 30, about 25, about 20, about 16, 15, 14, 13, 12, 11, 10, 9, 8, 7, 6, 5, or 4.

The assay electrodes 220A may be selected to be spaced according to a specified scheme. The specified scheme may be heterogenous or homogeneous. The assay electrode schemes may be any one or a combination of the following:
A fixed point along the axial plane of the chest along the reserve electrodes is selected, then the assay electrodes are selected at defined intervals around the perimeter from the fixed point. For example, the center of the sternum or the center of the spine may be the fixed point. The assay electrodes may then selected as those reserve electrodes located at defined percentages around the chest, starting from the fixed point, e.g., 25% around the chest starting from the center of the sternum, 50% around the chest starting from the center of the sternum, 75% around the chest starting from the center of the sternum, and 100% around the chest starting from the center of the sternum.
The assay electrodes may be equally spaced.
The assay electrodes may be symmetrical along the saggital plane of the chest.
The assay electrodes may be symmetrical along the coronal plane of the chest.
The assay electrodes may be asymmetrically spaced.
The assay electrodes may be irregularly spaced.
The location of each assay electrode may be manually selected by the user.

The assay electrodes 220A may be selected such that they are equally spaced along the device 205.

The location of the assay electrodes are set to be as close to the specified scheme as practically possible, given the number of reserve electrodes R. If the number of reserve electrodes is lower, it is typically more difficult to select assay electrodes that are located exactly at the location defined by a scheme. In such a case, the electrode located closest to the location specified by the scheme may be selected.

As an example, equal spacing is possible only in certain cases, e.g., where the number of reserve electrodes R is divisible by the number of assay electrodes A. For example, if there are 80 reserve electrodes, the system may select every 10 electrodes to achieve a desired 8 electrodes. However, in many cases, the number of reserve electrodes R may not be divisible by the number of assay electrodes A. In such a case, various solutions are possible for achieving the closest match to true equal spacing (which are also considered equal spacing for the purposes of the disclosure). First, as described above, the electrode located closest to the location specified by the scheme may be selected. Alternatively, a first pair of assay electrodes may be separated by a distance that is substantially different from the spacing of the rest of the assay electrodes. The distance between the first assay electrode pair may be selected such that the number of the remaining assay electrodes is divisible by the number of the remaining reserve electrodes.

Referring now to FIG. 6B, the device 205 may include a band 210 capable of maintaining the relative distances between the electrodes while the total length is adjustable. The device 205 may comprise only assay electrodes. Such a device may have a band 110 that is constructed of a stretchable material such that (1) the circumference of the devices adjusts to fit the circumference of the chest; and (2) the proportional location of the electrodes from each other is maintained. Alternatively, the band 110 may be constructed of a non-stretching material that is arranged in a set of mechanical contraptions, such as strap adjustors, such that (1) the circumference of the devices adjusts to fit the circumference of the chest; and (2) the proportional location of the electrodes from each other is maintained. The band 210 shown in FIG. 6B is a strip-type. It will be appreciated that the band may alternatively be a loop-type.

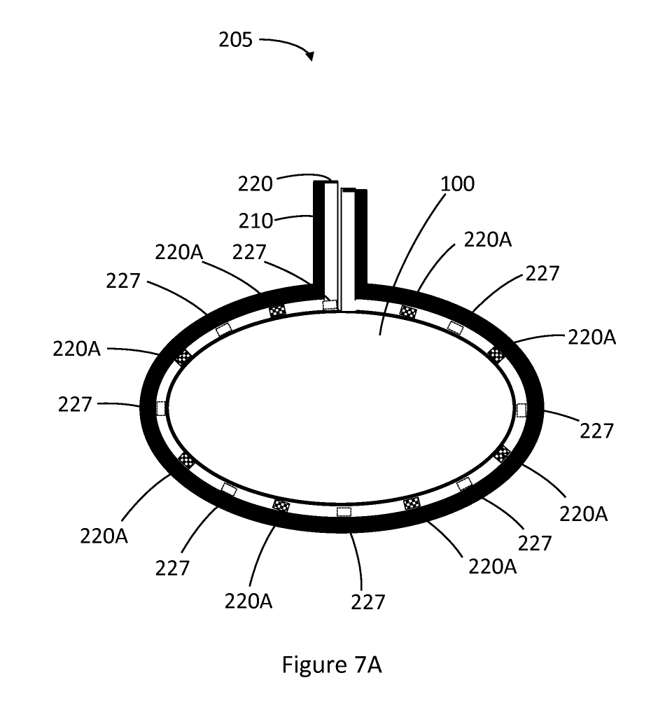

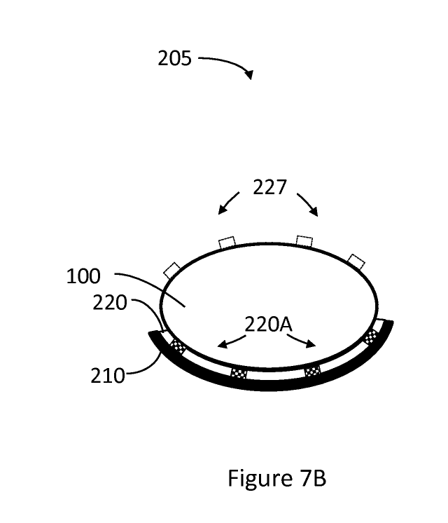

Referring now to FIGS. 7A and 7B, the device 205 may be configured to generate virtual electrodes 225. A virtual electrode 227 is not a physical electrode (i.e., one of the array of electrodes 220). Rather, the virtual electrode is a computationally generated construct, which allows for the analysis of the voltage recordings made in the assay electrodes 220A to be as if additional assay electrodes were in place. The virtual electrodes 227 may be configured such that they appear to be interleaved with the assay electrodes 220A, as shown in FIG. 7A. Alternatively or in combination, the virtual electrodes may be configured such that they appear to be located on the opposing side of the chest, as shown in FIG. 7B.

With particular reference to FIG. 7B, it is noted that a band having four physical electrodes 220A may be used even when it is of a length insufficient to completely encompass the chest of a patient. For example, four additional virtual electrodes 227 may be introduced.

### Method of Selecting Assay Electrodes

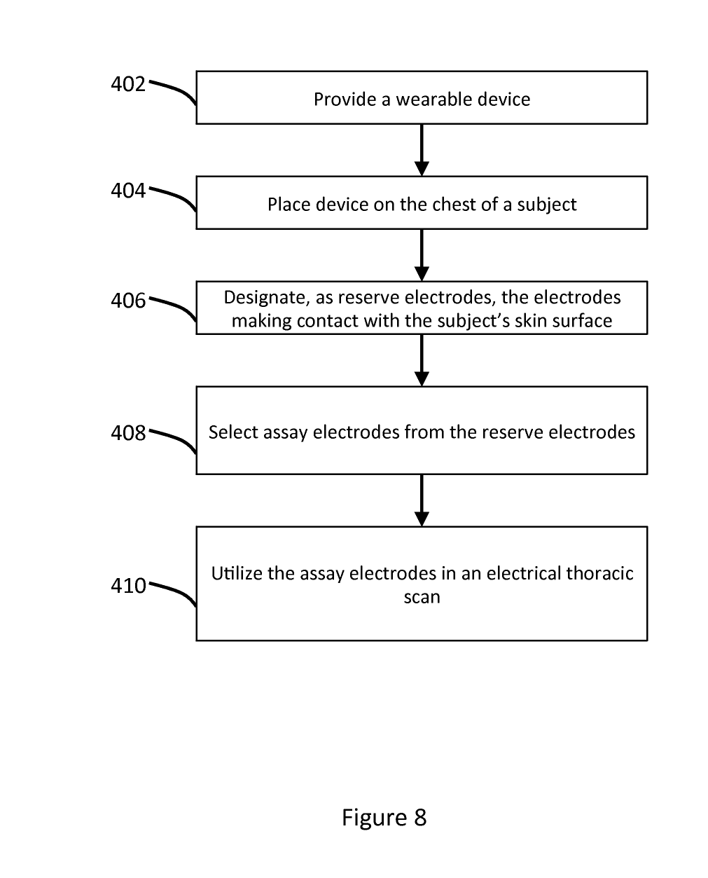

Reference is now made to FIG. 8, which is a flowchart showing a method of selecting one or more assay electrodes for use in a device for use in an electrical thoracic scan system, the device having a linear multielectrode array. The various options described for the system 200, the device 205, the device 1205, and their components as described with reference to FIGS. 1-7 are also options for the methods described in FIGS. 8-12.

The method may follow the following steps:
Providing a device for use in an electrical thoracic scan system, the device comprising: a band having an outer surface and an inner surface; a linear array of electrodes arranged equally spaced along the length of the band and on said inner surface for contacting said skin surface; each electrode being selectively connectable to a control unit. (See step 402).
Placing the device on the subject. (See step 404).
Designating as reserve electrodes the electrodes making contact with the subject's skin surface. (See step 406).
Selecting assay electrodes from the reserve electrodes. (See step 408).
Utilizing the assay electrodes in an electrical thoracic scan process (See step 410).

The above method includes two steps of electrode selection (steps 406 and 408) to determine which of the electrodes in the device are used for the injecting of current and/or measuring of voltage on the skin surface. First, out of the total electrodes on the device, those making contact with the subject's skin surface are designated as reserve electrodes. Second, assay electrodes are selected from among the reserve electrodes. The assay electrodes are then utilized in an electrical thoracic scan process. The remaining electrodes are unused, and may be disabled. In certain cases, all of the electrodes on the device may be designated as reserve electrodes. Further, in certain cases, all of the reserve electrodes may be selected as assay electrodes.

The electrical thoracic scan may be electrical impedance tomography (EIT), electrocardiography (ECG), body surface mapping, and the like. The EIT may be parametric EIT (pEIT).

The assay electrodes may be selected according to a specified scheme. The specified scheme may be heterogenous or homogeneous. The assay electrode schemes may be any one or a combination of the following:
A fixed point on the chest along the axial plane of the reserve electroded is selected, then the assay electrodes are selected at defined intervals from the fixed point around the chest. For example, the center of the sternum or the center of the spine may be the fixed point. The assay electrodes may then selected as those reserve electrodes located at defined percentages around the chest, starting from the fixed point, e.g., 25% around the chest starting from the center of the sternum, 50% around the chest starting from the center of the sternum, 75% around the chest starting from the center of the sternum, and 100% around the chest starting from the center of the sternum.
The assay electrodes may be equally spaced.
The assay electrodes may be symmetrical along the saggital plane of the chest.
The assay electrodes may be symmetrical along the coronal plane of the chest.
The assay electrodes may be asymmetrically spaced.
The assay electrodes may be irregularly placed.
The location of each assary electrode may be manually selected by the user.

In the case of the electrical thoracic scan being EIT or pEIT, the step of utilizing may include the steps of: connecting in a controlled sequence, under control of the microprocessor running a first algorithm, pairs of the assay electrodes to the current source, and recording the resulting voltages measurements from the remaining assay electrodes; and analyzing the resulting voltage measurements to generate an impedance image of the subject's chest.

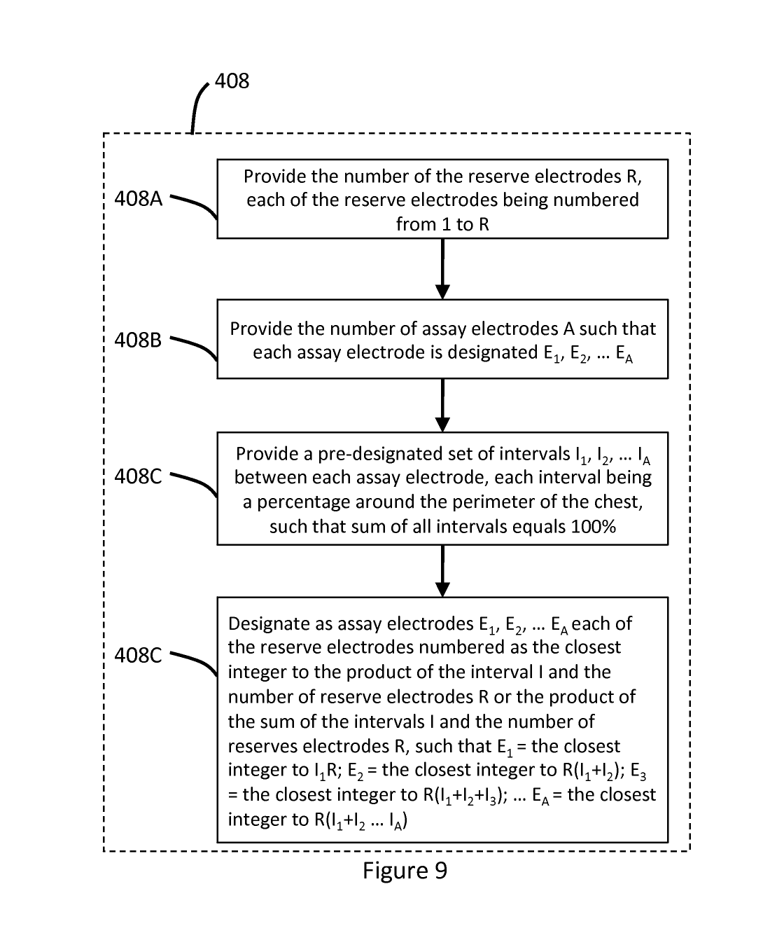

Reference is now made to FIG. 9, which is a flowchart showing an example of sub-steps for step 408 of the flowchart shown in FIG. 8 (the step of selecting assay electrodes from the reserve electrodes). The sub-steps of step 408 may be conducted under control of a microprocessor running an algorithm comprising the steps of:
providing the number of the reserve electrodes R, each of the reserve electrodes being numbered from 1 to R (sub-step 408A);
providing the number of assay electrodes A, such that each assay electrode is designated E₁, E₂, . . . E_{A} (sub-step 408B);
providing a pre-designated set of intervals I₁, 1₂, . . . I_{A} between each assay electrode, each interval being a percentage around the perimeter of the chest, such that sum of all intervals equals 100% (sub-step 408C);
designating as assay electrodes E₁, E₂, . . . E_{A} each of the reserve electrodes numbered as the closest integer to the product of the interval I and the number of reserve electrodes R or the product of the sum of the intervals I and the number of reserves electrodes R, such that E₁=the closest integer to I₁R; E₂=the closest integer to R(I₁+I₂); E₃=the closest integer to R(I₁+I₂+I₃); . . . E_{A}=the closest integer to R(I₁+I₂ . . . I_{A}) (sub-step 408A).

For example, if there are 100 reserve electrodes (R=100), and 5 assay electrodes are desired at predefined intervals I of 10%, 20%, 20%, 25% and 25%, then, along the array of reserve electrodes numbered 1 to 100, the electrodes numbered 10, 30, 50, 75 and 100 are selected to be the assay electrodes.

In certain assays, e.g., in the case of the electrical thoracic scan being EIT or pEIT, the assay electrodes are preferably equally spaced.

As discussed above, equally spaced means equally spaced as practically possible, given the number of reserve electrodes R and the number of assay electrodes A. True equal spacing is possible only in certain cases, e.g., where the number of reserve electrodes R is divisible by the number of assay electrodes A. For example, if there are 80 reserve electrodes, the system may select every 10 electrodes to achieve a desired 8 electrodes. However, in many cases, the number of reserve electrodes R may not be divisible by the number of assay electrodes A. In such a case, various solutions are possible for achieving the closest match to true equal spacing (which are also considered equal spacing for the purposes of the disclosure).

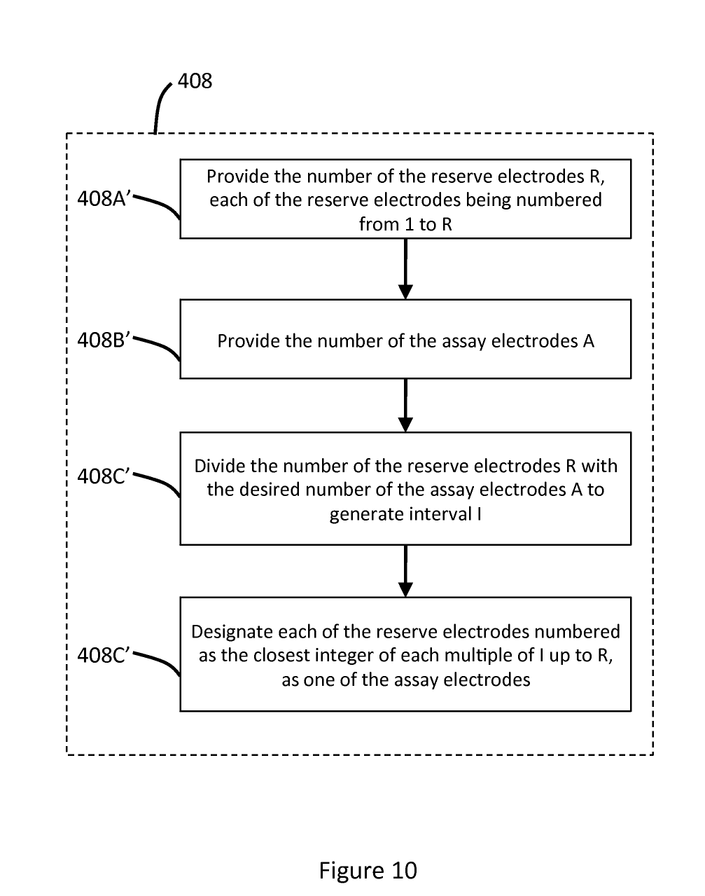

Reference is now made to FIG. 10, which is a flowchart showing another example of sub-steps for step 408 of the flowchart shown in FIG. 8 (the step of selecting assay electrodes from the reserve electrodes). The sub-steps of step 408 may be conducted under control of a microprocessor running an algorithm comprising the steps of:
providing the number of the reserve electrodes R, each of the reserve electrodes being numbered from 1 to R (sub-step 408A′);
providing the number of assay electrodes A (sub-step 408B′);
   dividing the number of the reserve electrodes R with the desired number of the assay electrodes A to generate interval I (sub-step 408C′); and
designating each of the reserve electrodes numbered as the closest integer of each multiple of I up to R, as one of the assay electrodes (sub-step 408D′).

Further, the position of the assay electrodes may be shifted by adding or subtracting a shifting value S.

For example, if there are 105 reserve electrodes (R=105), and 8 assay electrodes are desired, then the algorithm provides an interval I of 13.125 (i.e. 105/8). As such, along the array of reserve electrodes numbered 1 to 105, the electrodes numbered 13, 26, 39, 53, 66, 79, 92, 105 are selected to be the assay electrodes.

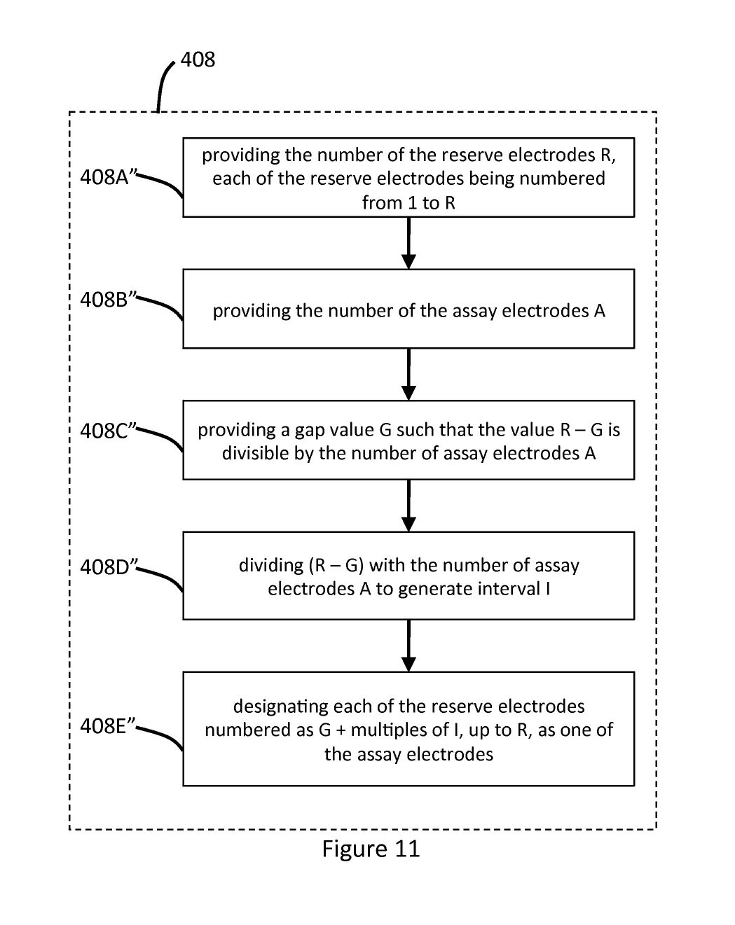

Reference is now made to FIG. 11, which is a flowchart showing another example of sub-steps for step 408 of the flowchart shown in FIG. 8 (the step of selecting assay electrodes from the reserve electrodes). The substeps of step 408 may be conducted under control of a microprocessor running an algorithm comprising the steps of:
providing the number of the reserve electrodes R, each of the reserve electrodes being numbered from 1 to R (sub-step 408A″);
providing the number of assay electrodes A (sub-step 408B″);
providing a gap value G such that the value R−G is divisible by the number of assay electrodes A (sub-step 408C″);
dividing (R−G) with the number of assay electrodes A to generate interval I (sub-step 408D″); and
designating each of the reserve electrodes numbered as G +multiples of I, up to R, as one of the assay electrodes (sub-step 408E″).

Further, the position of the assay electrodes may be shifted by adding or subtracting a shifting value S.

Using the same example as above, of there being 105 reserve electrodes (R=105), and 8 equally spaced assay electrodes being desired. If G is selected to be 25, then (R−G) is 80, which results in an interval I of 10 (i.e. 80/10). As such, along the array of reserve electrodes numbered 1 to 105, the electrodes numbered 35, 45, 55, 65, 75, 85, 95, and 105 are selected to be the assay electrodes.

Each of the above two algorithms to select equally spaced assay electrodes have advantages and disadvantages. The algorithm shown in FIG. 10 may result in some unevenness of the distribution of the assay electrodes. However, this unevenness is negligible in cases where the number of reserve electrodes R is substantially larger than the number of assay electrodes A. The algorithm shown in FIG. 11 may result in one pair of assay electrodes being separated by a distance that is substantially different from the spacing of the rest of the assay electrodes. However, such an arrangement may be preferable according to the needs of the system, for example, when the number of reserve electrodes is small or not substantially greater than the number of assay electrodes A.

For example, if the number of reserve electrodes is 13, and the number of assay electrodes is 8, the assay electrodes assigned according to the algorithm shown in FIG. 10 would be 2, 3, 5, 6, 8, 9, 11 and 12, while the assay electrodes assigned according to the algorithm shown in FIG. 11 can be 5, 6, 7, 8, 9, 10, 11 and 12 (with a gap value of 4). As such, the algorithm shown in FIG. 11 may be preferable in this case, where with the exception of one pair of electrodes, the rest are spaced with perfectly equal spacing.

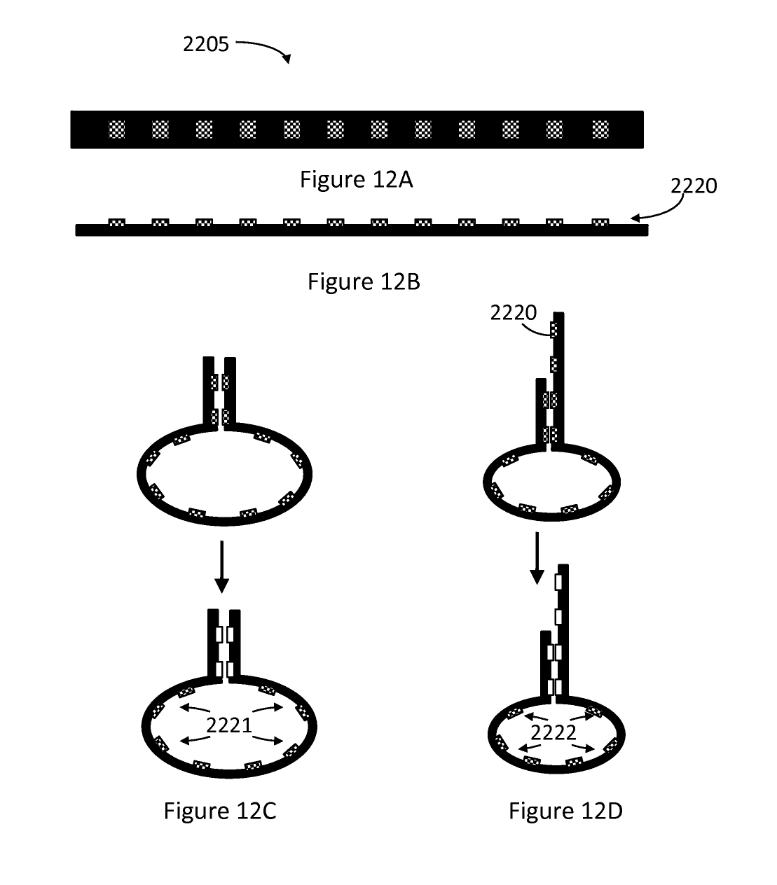

Referring now to FIGS. 12A-12D, a similar outcome as the algorithm shown in FIG. 11 may arise in the case of a device 2205 containing a low number of electrodes 2220, e.g., 12 electrodes (see FIGS. 12A-12B). FIG. 12C represents a case where the device 2205 fully encircles the chest 100 such that 8 electrodes 2221 are contacting the skin surface. These 8 electrodes are designated as reserve electrodes, and all 8 reserve electrodes are selected as assay electrodes. Depending of the circumference of the chest, the distance of the two electrodes closest to the juxtaposition of the device may be different (more or less) from the spacing between the remaining electrodes. For the purposes of this disclosure, such an arrangement of assay electrodes is considered to be equally spaced. FIG. 12D shows a similar situation with a smaller chest, with 6 reserve electrodes 2222 and all 6 reserve electrodes being selected as assay electrodes.

The scope of the disclosed embodiments may be defined by the appended claims and includes both combinations and sub combinations of the various features described hereinabove as well as variations and modifications thereof, which would occur to persons skilled in the art upon reading the foregoing description.

Technical and scientific terms used herein should have the same meaning as commonly understood by one of ordinary skill in the art to which the disclosure pertains. Nevertheless, it is expected that during the life of a patent maturing from this application many relevant systems and methods will be developed.

As used herein the term “about” refers to at least about 10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A method of selecting one or more assay electrodes for use in a device for use in an electrical thoracic scan system, the device having a linear multielectrode array, the method comprising the steps of:
providing a device for use in an electrical thoracic scan system, the device comprising: a band having an inner surface; a linear array of electrodes arranged equally spaced along the length of the band and on said inner surface for contacting said skin surface; each electrode being selectively connectable to a control unit;
placing said device on the chest of the subject;
designating, as reserve electrodes for potential use, the electrodes making contact with the skin surface;
selecting a plurality of assay electrodes from said reserve electrodes; and
utilizing the assay electrodes in an electrical thoracic scan process.

2. The method of claim 1 further comprising at least one step selected from a group consisting of (a) disabling the electrodes not in contact with the skin surface; (b) partially encircling the chest by said band; and any combination thereof

3. The method of claim 1, wherein the step of placing said device on the chest of the subject comprises the sub-step of wrapping said device fully around the chest of the subject such that:
at least a portion of the band fully encircles the chest;
the electrodes in the portion of the band fully encircling the chest are in contact with the skin surface;
the electrodes in the remaining portion of the band, if present, are not in contact with the skin surface.

4. The method of claim 3, wherein the step of placing said device on the chest of the subject further comprises the sub-step of:
securing the fully wrapped device at the point of band juxtaposition with a connector configured to disable the electrodes in said remaining portion of the band.

5. The method of claim 3, wherein the device is configured to measure the circumference of the chest of the subject.

6. The method of claim 3, wherein the circumference of the chest of the subject is measured by a method comprising the steps of:
passing an electric current through a section of a wire running through the length of the device corresponding to the portion of the band fully encircling the chest; and
calculating the circumference based on the voltage difference through said section of the wire,
wherein the wire is characterized by a known resistance per unit length.

7. The method of claim 3, wherein the outer surface of the device comprises a plurality of optical patterns corresponding to length values, wherein the circumference of the chest of the subject is measured by a method comprising the steps of:
reading, via an optical reader, two optical patterns closest to each side of the point of juxtaposition of the device around the chest; and
calculating the circumference based on the difference of the length values corresponding to the two optical patterns read by the optical reader.

8. The method of claim 3, wherein the circumference of the chest of the subject is measured by a method comprising the steps of:
providing the number of reserve electrodes; and
calculating the chest circumference by multiplying the number of reserve electrodes with the inter-electrode distance.

9. The method of claim 1, wherein the electrical thoracic scan is selected from the group consisting of: electrical impedance tomography (EIT), parametric EIT (pEIT), electrocardiography (ECG) and body surface mapping.

10. The method of claim 1, wherein the assay electrodes are selected according to at least one of the specified scheme selected from the group consisting of:
a fixed point on the chest along the axial plane of the reserve electroded is set and the assay electrodes are selected at defined intervals from the fixed point around the chest;
the assay electrodes are equally spaced;
the assay electrodes are symmetrical along the saggital plane of the chest;
the assay electrodes are symmetrical along the coronal plane of the chest;
the assay electrodes are asymmetrically spaced;
the assay electrodes are irregularly spaced;
the assary electrode are manually selected.

11. The method of claim 10, wherein the assay electrodes are designated under control of the microprocessor running a second algorithm comprising the steps of:
providing the number of the reserve electrodes R, each of the reserve electrodes being numbered from 1 to R;
providing the number of assay electrodes A, such that each assay electrode is designated E₁, E₂, . . . E_{A};
providing a pre-designated set of intervals I₁, I₂, . . . I_{A} between each assay electrode, each interval being a percentage around the perimeter of the chest, such that sum of all intervals equals 100%;
designating, as assay electrodes E₁, E₂, . . . E_{A}, each of the reserve electrodes numbered as the closest integer to the product of the interval I and the number of reserve electrodes R or the product of the sum of the intervals I and the number of reserves electrodes R, such that E₁=the closest integer to I₁R; E₂=the closest integer to R(I₁+I₂); E₃=the closest integer to R(I₁+I₂+I₃); . . . E_{A}=the closest integer to R(I₁+I₂ . . . I_{A}).

12. The method of claim 10, wherein the assay electrodes are equally spaced.

13. The method of claim 12, wherein the equally spaced assay electrodes are designated under control of the microprocessor running a second algorithm comprising the steps of:
providing the number of the reserve electrodes R, each of the reserve electrodes being numbered from 1 to R;
providing the number of assay electrodes A;
dividing the number of the reserve electrodes R with the desired number of the assay electrodes A to generate interval I;
designating each of the reserve electrodes numbered as the closest integer of each multiple of I up to R, as one of the assay electrodes.

14. The method of claim 12, wherein the equally spaced assay electrodes are designated under control of the microprocessor running a third algorithm comprising the steps of:
providing the number of the reserve electrodes R, each of the reserve electrodes being numbered from 1 to R;
providing the number of assay electrodes A;
providing a gap value G such that the value R−G is divisible by the number of assay electrodes A;
dividing (R−G) with the number of assay electrodes A to generate interval I; and
designating each of the reserve electrodes numbered as multiples of I+G, up to R, as one of the assay electrodes.

15. The method of claim 1, wherein at least one of the following is being held true (a) the number of electrodes E is more than 50, more than 100, more than 150, more than 200, more than 300, between 50 and 300, between 100 and 300, or between 100 and 500; (b) the electrodes are integrated into a printed circuit board; (c) the device is disposable; and any combination thereof.

16. The method of claim 1, additionally comprising a step of integrating the device into an article of clothing, said article of clothing is selected from the group consisting of: a belt, a shirt, a vest and a bra.

17. A device for use in an electrical thoracic scan system, the device comprising:
a band having an outer surface and an inner surface;
a linear array of electrodes spaced along the length of the band and on said inner surface for contacting said skin surface;
wherein each electrode is selectively connectable to a control unit.

18. The device of claim 17, wherein at least one of the following is being held true (a) the electrical thoracic scan is selected from the group consisting of: electrical impedance tomography (EIT), parametric EIT (pEIT), electrocardiography (ECG) and body surface mapping; (b) the electrical thoracic scan is EIT or pEIT; (c) each electrode is selectively connectable to a current source unit or a voltage measurement unit and said current source unit and voltage measurement unit are independently controlled by a microprocessor, said set of selectively connectible electrodes comprising a set of assay eletrodes, such that pairs of the assay electrodes are connectable to the current source, in a controlled sequence, under control of the microprocessor, and the resulting voltages measurements from the remaining assay electrodes are analyzable to generate an impedance image of the subject's chest; and any combination thereof; (d) at least a portion of the band is configured to fully encircle the chest; (e) the electrodes in the portion of the band fully encircling the chest are in contact with the skin surface; and the electrodes in the remaining portion of the band, if present, are not in contact with the skin surface; (f) the electrodes are integrated into a printed circuit board; (g) the device is disposable; and any combination thereof.

19. The device of claim 18, wherein the device is integrated into an article of clothing; further wherein the article of clothing is selected from the group consisting of: a belt, a shirt, a vest and a bra.

20. The device of claim 18, wherein the assay electrodes are selected according to at least one of the specified schemes selected from a group consisting of:
a fixed point on the chest along the axial plane of the reserve electroded is set and the assay electrodes are selected at defined intervals from the fixed point around the chest;
the assay electrodes are equally spaced;
the assay electrodes are symmetrical along the saggital plane of the chest;
the assay electrodes are symmetrical along the coronal plane of the chest;
the assay electrodes are asymmetrically spaced;
the assay electrodes are irregularly spaced;
the assary electrode are manually selected.
